(19) United States
(12) Patent Application Publication
(10) Pub. No.: US 2022/0195056 A1
(43) Pub. Date: Jun. 23, 2022

(54) METHOD OF TREATING KELOIDS

(71) Applicant: Icahn School of Medicine at Mount Sinai, New York, NY (US)

(72) Inventors: Emma Guttman-Yassky, New York, NY (US); Ana Brandusa PAVEL, New York, NY (US)

(73) Assignee: Icahn School of Medicine at Mount Sinai, New York, NY (US)

(21) Appl. No.: 17/633,255

(22) PCT Filed: Aug. 6, 2020
(86) PCT No.: PCT/US2020/045176

§ 371 Date: Feb. 7, 2022

Related U.S. Application Data
(60) Provisional application No. 62/884,119, filed on Aug. 7, 2019.
(60) Provisional application No. 62/938,709, filed on Nov. 21, 2019.

(51) Int. Cl. C07K 16/28; C07K 16/24; A61K 31/4985; A61K 31/519; A61K 31/506; A61K 31/541; A61K 31/437; A61P 17/02

(52) U.S. Cl. CPC C07K 16/2866; C07K 16/244; A61K 31/4985; A61K 31/519; A61K 2039/505; A61K 31/541; A61K 31/437; A61P 17/02; A61K 31/506

(57) ABSTRACT

The present disclosure relates to methods for diagnosing and staging keloids, and to methods of treating keloids using therapeutic compositions that inhibit the Th2 cytokine signaling pathway.

### RELATED APPLICATIONS

This application is a § 371 national stage of PCT International Application No. PCT/US20/45176, filed Aug. 6, 2020, the application claims priority to U.S. Provisional Application No. 62/884,119 filed Aug. 7, 2019 and U.S. Provisional Application No. 62/938,709 filed Nov. 21, 2019, the contents of which are incorporated herein by reference in their entirety.

### FIELD OF THE INVENTION

The present invention relates generally to methods of diagnosing, staging, and treating keloids and to methods of regulating Th2 immune activity in multiple inflammatory diseases.

### BACKGROUND

Keloids are benign growths characterized by an abnormal healing process that involves excessive collagen proliferation and degradation. Keloid lesions grow over time, often recur following therapy, can spread from the site of origin, and do not regress spontaneously. The clinical manifestations can cause considerable discomfort, pain and pruritus, which are often associated with significant psychosocial impairment, disfiguration, reduced mobility and an overall reduced quality of life (Madu & Kundu, 2014). Keloid scarring has a strong familial heritability component, with a higher incidence in individuals with dark, pigmented, ethnic skin of African, Asian, and Hispanic descent (Ud-Din & Bayat, 2013).

To date, multiple therapies with limited success rates have been proposed for the treatment of keloids, including intralesional steroids, fluorouracil, bleomycin, surgical excision, laser therapy, radiation, cryotherapy, botulinum toxin, etc. (Ud-Din & Bayat, 2013). The lack of treatment options stems largely from the limited molecular profiling of keloids and paucity of understanding of pathological mechanisms. Most prior work in keloids primarily focused on the connective tissue abnormalities. A recent paper using transcriptional profiling in lesional and non-lesional skin biopsies obtained from three African American patients with chronic keloids, identified >1,200 upregulated differentially expressed genes between chronic keloid tissues and non-lesional skin from keloid patients (Fuentes-Duculan et al., 2017). These genes included genes involved in wound healing response and bone/cartilage formation, including fibrillin 2, asporin, cadherin 11, and runt-related transcription factor 2, to name a few; however this profiling study did not evaluate inflammatory pathways.

Thus, there remains a very high unmet need for more effective therapeutics for keloids, which also pose major challenges due to their high recurrence rates after current treatments. The present invention addresses these deficiencies.

### SUMMARY OF THE INVENTION

The present invention identifies for the first time a therapeutic keloid response to dupilumab, which blocks type 2-driven inflammation via IL-4 and/or IL-13 signaling. This discovery reveals an underlying Th2 pathogenesis for keloid formation and illuminates methods and pathways for the treatment of chronic keloids. The present invention further shows the efficacy of inhibiting Th2 cytokines and inflammatory pathways as methods for treating keloids.

One aspect of the present invention provides biological markers that are directly linked with keloids in tissues.

Another aspect of the present invention provides biological markers for staging or tracking keloid pathogenesis or response to treatment.

Another aspect of the present invention provides a biological marker and a method for treating keloids.

Another aspect of the present invention provides a method of screening for compositions and methods to treat or prevent keloids.

Another aspect of the present invention provides compositions for treating a keloid by inhibiting inflammatory pathways.

Another aspect of the present invention provides methods for treating keloids using an antibody.

Another aspect of the present invention provides methods for treating keloids using an antibody that interferes with both IL-4 and IL-13 signaling, or IL-13 or IL-4 signaling alone.

Another aspect of the present invention provides methods for treating keloids by inhibiting a cytokine pathway.

Another aspect of the present invention provides methods for characterizing keloids using Type 2 chemokines, such as CCL18, CCL11, CCL25, Periostin (POSTN), and other Th2 associated markers, such as OX40, OX40L, JAK3, IL-33, TSLP, and IL-5 expression levels.

Another aspect of the present invention provides methods for characterizing keloids using IL-4 and IL-4R expression levels.

Another aspect of the present invention provides methods for characterizing keloids using IL-13 expression levels.

Another aspect of the present invention provides methods for treating keloids by interfering with IL-4 signaling.

Another aspect of the present invention provides methods for treating keloids by interfering with IL-13 signaling.

Another aspect of the present invention provides methods for treating keloids by interfering with the Th2 signaling pathway.

### BRIEF DESCRIPTION OF THE FIGURES

FIG. 1 shows the clinical findings of pre- vs post-dupilumab treatment and inflammatory biomarker expression. (A) On the right popliteal fossa, a large depigmented keloid (denoted by the dotted arrow) measured at 3.5 cm horizontal, 2.8 cm vertical, and 2.1 cm, and a smaller adjacent keloid (denoted by the bold arrow) measured at 1.2 cm horizontal, 0.7 cm vertical, and 1.1 cm, August 2018. (B) Following initiation of dupilumab treatment, same lesional areas were evaluated and the larger isolated nodule measured at 1.6 cm horizontal, 1 cm vertical, and 0.9 cm (dotted arrow), with complete disappearance of the smaller adjacent keloid (bold arrow). Images to scale and taken with different perspectives. Th2-specific inflammatory biomarkers (C-E) measured by quantitative real-time PCR in healthy skin and keloid non-lesional (NL) and lesional (LS) skin. Black stars: significance of comparison between keloid skin and controls. Black lines within boxes represent median values; bold red lines represent mean values. Each black dot represents an individual patient; ⁺P<0.1, *P<0.05.

FIG. 2 shows the gene expression of keloid-specific biomarkers. Cartilage and bone-related markers (A-H) measured by quantitative real-time PCR in healthy skin and keloid non-lesional (NL) and lesional (LS) skin. Black stars: significance of comparison between keloid skin and controls; red stars: significance of comparison between lesional vs. non-lesional keloid skin. Black lines within boxes represent median values; bold red lines represent mean values. Each black dot represents an individual patient; ⁺P<0.1, *P<0.05, **P<0.01, ***P<0.001.

FIG. 3 shows the clinical findings of keloid response after 3 months of treatment with dupilumab (dupixent), an anti-IL-4R blocker that blocks signaling through IL-4 and IL-13 cytokines. (A) The image depicts keloids before treatment with dupilumab in an African American patient. (B) The image depicts keloids in an African American patient after 1.5 months of treatment with dupilumab (subcutaneous injections of 300 mg every week, after 600 mg induction at baseline). (C) The image depicts keloids in the same patient at the end of the third month of treatment with dupilumab. There is a noticeable reduction in the keloid size particularly in the height of the protrusion from the epithelium, but also in the diameter.

FIG. 4 shows the clinical findings of keloid response after 3 months of treatment. The image depicts another large keloid in a different African American than the one provided in FIG. 3. The image on the left shows the keloid prior to treatment, and the one on the right shows the keloid after 3 months of treatment (weekly subcutaneous injections of 300 mg, after a 600 mg induction at baseline). The keloid shrank significantly in diameter (2 cm) and in height (0.5 cm) as seen from the wrinkling of the surface denoting the vast shrinkage in all dimensions.

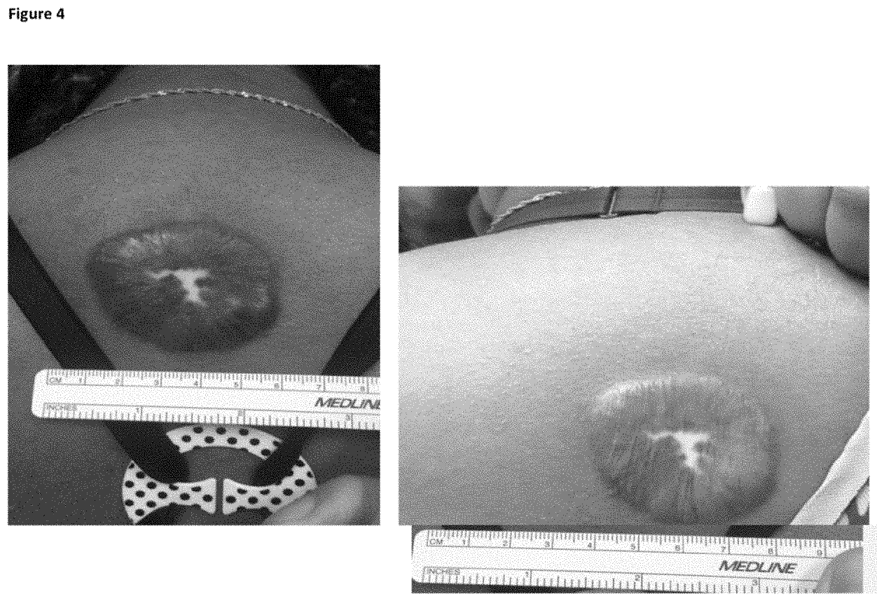

FIG. 5 shows the clinical findings of keloid response after 3 months of treatment. The image depicts another large keloid in a different patient than those shown in FIGS. 3 & 4. The image on the left shows the keloid prior to treatment, and the one on the right shows the keloid after 3 months of treatment (weekly subcutaneous injections of 300 mg). The keloid shrank significantly in diameter (0.8 cm) and in height (0.5 cm), and there is a noticeable wrinkling of the surface denoting the vast shrinkage in all dimensions.

FIG. 6 expression levels of many proteins evaluated in serum of keloid patients, compared to controls, as well as to patients with atopic dermatitis and psoriasis, two disease known to have a high level of systemic inflammation in the circulation. Proteomic data using OLINK Proseek platform shows IL4 to be increased in keloids blood compared to controls (p<0.1), further supporting the efficacy of inhibiting Th2 cytokines and inflammatory pathways as methods for treating keloids. This data also shows that levels of IL-4 in keloids are even higher than atopic dermatitis, that is considered a Th2 disease. These data also show upregulations in serum in other Th2 markers in keloid patients, such as IL-33, TSLP, and IL-5.

FIG. 7 shows proteomic data in serum of keloid patients, compared to controls, as well as psoriasis and atopic dermatitis patients Proteomic data using OLINK shows much higher levels of cardiovascular makers in keloids blood compared to controls, but also compared to psoriasis and atopic dermatitis patients, disease previously associated with increases in cardiovascular associated markers. (+p<0.1, *p<0.05, **p<0.01).

FIG. 8 shows mRNA expression of several Th2 related markers in skin of keloid patients by RT-PCR. The tested markers include JAK3, OX40 and OX40L.

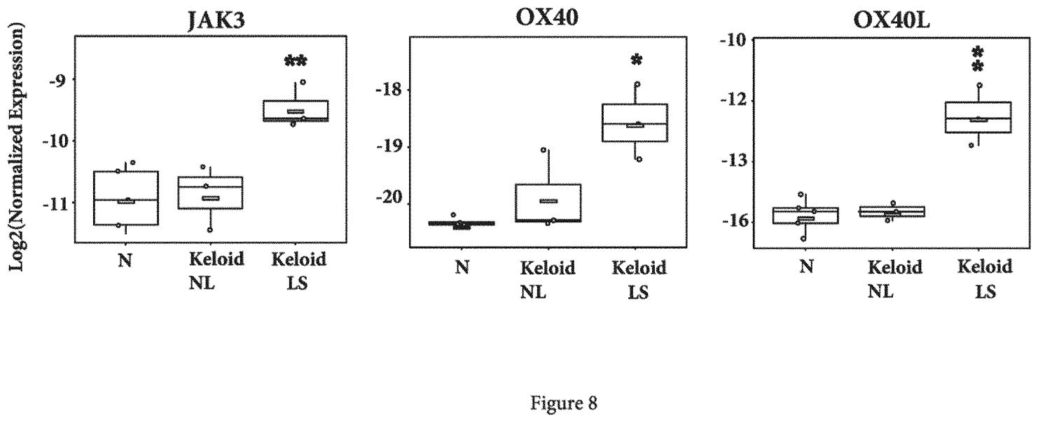

FIG. 9 shows significant changes in immune markers in lesional keloid skin compared to controls by RNA sequencing. This data strengthens the inventors findings of Th2 activation with many Th2 cytokines and chemokines that are part of Th2 pathway being significantly upregulated in keloids (IL4R and CCL11, TNFRSF4/OX40, TNFSF4/OX40L, CCL4, IL7R/TSLPR, CCL25), upregulation of innate immunity (IL6), Th17 (PI3, S100A8, S100A9, S100A12, CCL20) and JAK signaling (JAK3), as well as downregulation of IL34 and IL37 negative regulators (+p<0.1, *p<0.05, **p<0.01).

### DETAILED DESCRIPTION

Note that the term “subject” in the present invention is not particularly limited, and examples thereof include humans, mice, rats, cattle, horses, pigs, sheep, monkeys, dogs, and cats.

The term “therapeutic composition” according to the present invention may be in the form of an antibody, antibody fragment, antibody conjugate, vaccine, adjuvant, biological, pharmaceutical composition, a reagent used in an animal model, or a combination of such ingredients. The antibody, antibody fragment, antibody conjugate, vaccine, adjuvant, biological, pharmaceutical composition, or reagent, or combinatorial product can have the effect of reducing or eliminating keloids in a subject. Administration of such therapeutic compositions may be topical, oral, buccal, or parenteral.

The term “Th2” means T-helper type 2, which help regulate immune responses by releasing cytokines. The terms “IL-13” and “IL-4” (both of which are Th2 cytokines) refer to interleukin 13 and Interleukin 4 as is understood by persons of skill in the art. The term “Type 2 chemokine” includes, but is not limited to CCL17, CCL18 (or chemokine ligand 18) and CCL22.

The term “dupilumab” refers to a fully human monoclonal antibody that targets IL-4 receptor α (IL-4Rα), the shared subunit of the type 2 cytokines IL-4 and IL-13 and inhibits signaling of both Type 2 cytokines. Dupilumab is approved in an every other week dosing in the United States for the treatment of atopic dermatitis in adults (300 mg every other week)) and adolescents above 12 years of age (200/300 mg every other week) and has completed phase 3 studies in 6-11 years old children with atopic dermatitis. Dupilumab is registered with the FDA under UNII 420K487FSG. The term “lebrikizumab” refers to a humanized monoclonal antibody directed against IL-13, registered with the FDA under UNII U9JLP7V031. The term “tralokinumab” refers to a fully human monoclonal antibody directed against IL-13 that is under investigation for the treatment of atopic dermatitis. Tralokinumab is registered with the FDA under UNII GK1LYB375A.

### EXAMPLES

Provided below are select examples of certain embodiments of the present invention; however, the invention is not limited to these examples or the specific embodiments recited above.

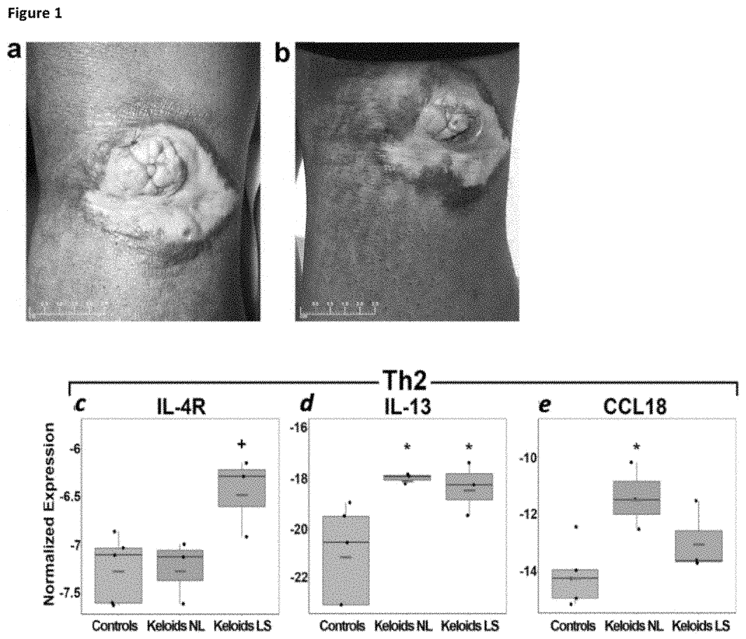

An African American subject afflicted with severe atopic dermatitis (AD) (body surface area/BSA 70%; SCORing of AD/SCORAD, 50; Eczema Area and Severity Index/EASI, 33) post-inflammatory hypopigmentation, and two keloid nodules was treated with dupilumab. The subject exhibited a large prominent nodule with raised borders, and a smaller adjacent nodule on the right popliteal fossa (FIG. 1A). Both nodules were present for more than 2 years. Each was diagnosed histologically as a keloid, and prior treatment included intralesional triamcinolone injections, which resulted in minimal improvement. The subject received 300 mg subcutaneous dupilumab injections for severe AD, administered every 2 weeks for a month. The inventors surprisingly discovered that seven months after dupilumab treatment, the subject experienced drastic (>50%) reduction in size of the large keloid with flattening of surrounding borders, and complete disappearance of the smaller adjacent keloid (FIG. 1B).

Dupilumab is a fully human monoclonal antibody that targets IL-4 receptor α (IL-4Rα), the shared subunit of the type 2 cytokines IL-4 and IL-13 and inhibits signaling of both Type 2 cytokines. In view of dupilumab's activity, the inventors investigated the role of the Th2 signaling pathway in keloids. The inventors used real-time PCR to evaluate gene expression of Th2 markers related to IL-4R targeting (IL-4R, IL-13, CCL18) in lesional and non-lesional keloid skin from three previously reported AA patients (n=3, 3 females, mean age, 47.3) with severe chronic keloids and no concurrent AD. These were compared with results from five healthy AA controls (n=5, 2 females, 3 males, mean age, 39.8) were included for comparisons. Six-millimeter whole-skin biopsy specimens were obtained from extremities under IRB-approved protocols.

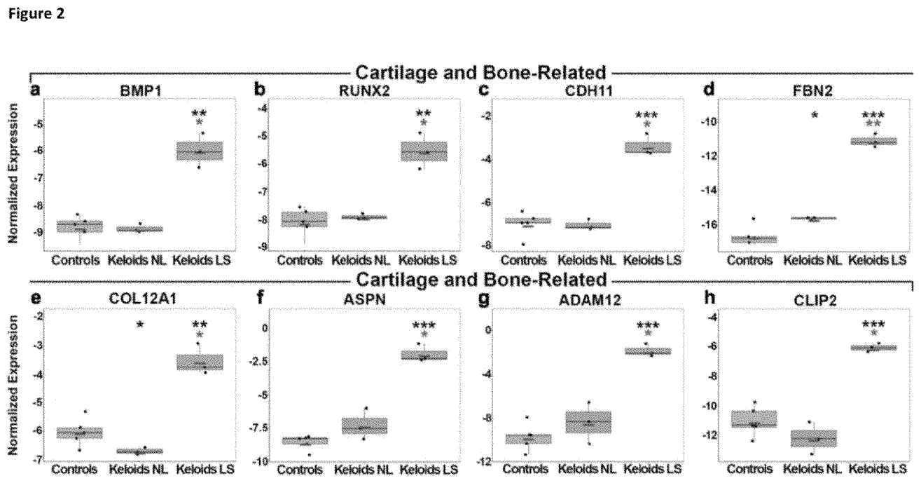

The inventors surprisingly discovered that IL-4R, directly targeted by dupilumab, was highly up-regulated in keloid lesions versus controls (P<0.1; FIG. 1C). IL-13, a key Th2 cytokine, was significantly increased in lesional and non-lesional keloids versus controls (P<0.05; FIG. 1D). Th2 chemokine, CCL18, was also highly increased in keloids, particularly in non-lesional skin (P<0.05; FIG. 1E). The inventors also evaluated genes involved in cartilage/bone development, including collagen type XII alpha 1, and cartilage intermediate layer protein 2 which were previously reported as highly expressed in keloids. They determined that all were significantly increased in keloid lesions versus controls and vs. non-lesional skin (P<0.05; FIG. 2).

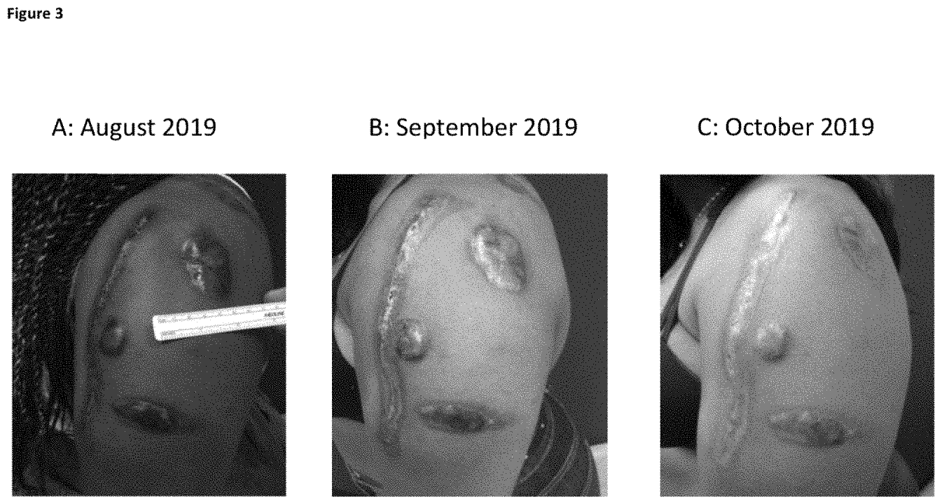

The inventors further discovered that treatment with dupilumab over a three-month period significantly reduced keloid size in an additional African American patient. An African American subject with keloids, but without other dermatological symptoms, was treated with dupilumab. The subject received 300 mg subcutaneous dupilumab injections, administered weekly. Comparisons of keloid sizes before treatment, 1.5 months after treatment, and 3 months after treatment reveal reduction in size and depth. There is a noticeable reduction in the keloid size, particularly in the height of the protrusion from the epithelium, but also in the diameter (FIGS. 3 & 4).

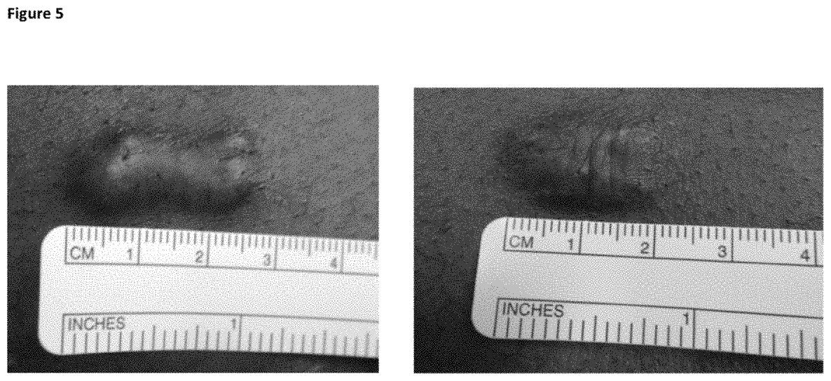

The inventors observed a significant keloid reduction in yet a third patient treated with dupilumab. Another African American patient with keloids, but no other dermatological symptoms, received weekly subcutaneous injections of dupilumab (300 mg) and observed a positive response. As seen in FIG. 5, there is a noticeable reduction in the keloid size, as observed by the reduction in height and length and in the wrinkling of the keloid.

The inventors discovered that injections of dupilumab every other week were insufficient to treat keloids. Surprisingly, every African American patient (three of the three patients treated) receiving weekly dupilumab injections observed a reduction in keloid size and shape. In addition, only the weekly injections were sufficient to maintain the size reduction and to treat the pain and irritation associated with the keloids in the afflicted patients.

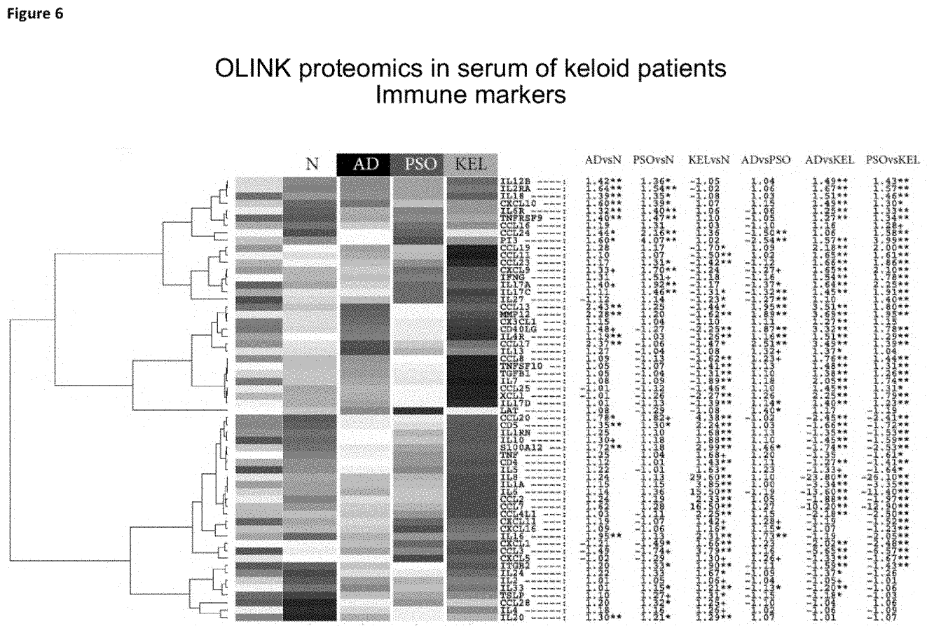

To better understand the immune markers involved in keloids, the inventors also examined immune markers in the serum of keloid patients using proteomics. The results of the study revealed an increase in IL-4 in the blood of patients afflicted with keloids compared to controls (p<0.1) and shows that IL-4 in keloids are higher than in patients with atopic dermatitis (FIG. 6). Prior work shows an increase in certain inflammatory and cardiovascular protein markers among patients afflicted with AD (Brunner et al. 2017). Notably, atopic dermatitis is considered a Th2 disease, further supporting the efficacy of inhibiting Th2 cytokines and inflammatory pathways as methods for treating keloids.

Atherosclerosis is known to be mediated by local inflammatory mediators including chemokines and their receptors, that are involved in the recruitment of inflammatory cells to the intima as an essential step in plaque development. For example, CCL4 and its receptor CCR5 have been demonstrated to play diverse roles in the inflammatory events underlying cardiovascular diseases and diabetes mellitus. CXCL5 is increased in atherosclerosis, mediating a protective role in a mouse model by modulating macrophage activation. CCL28 is chemotactic to T-cells, B-cells, and eosinophils to mucosal effector sites, and is increased in asthma. And CCL17 has been shown to drive atherosclerosis by restraining regulatory T-cell homeostasis, and CXCL10 is associated with the severity of coronary artery disease.

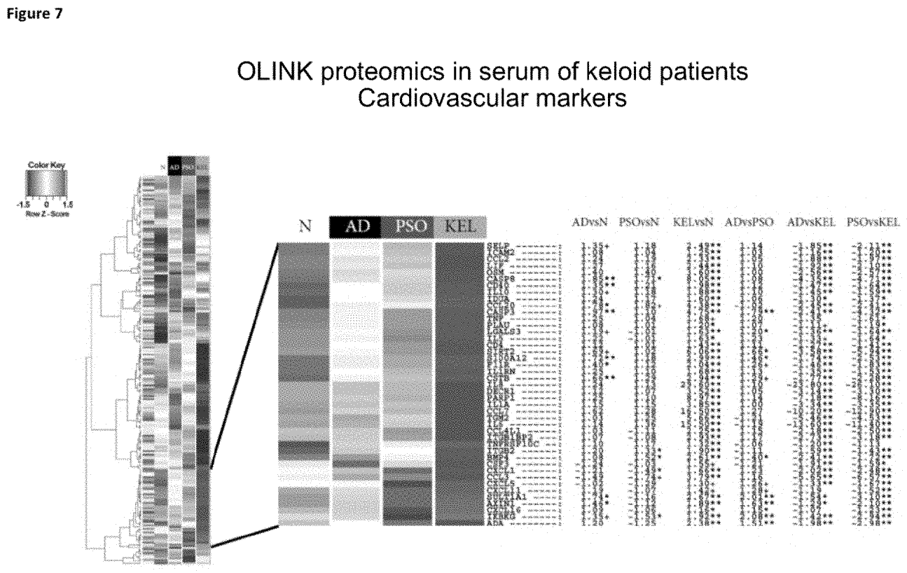

A examination of certain inflammatory markers in blood using proteomics in keloid patients versus controls, psoriasis, and atopic dermatitis patients also showed that keloid patients have much higher levels of certain cardiovascular markers compared to all three groups (controls +p<0.1, psoriasis, *p<0.05, atopic dermatitis **p<0.01) (FIG. 7). In combination, these results led the inventors to the remarkable discovery that keloids are associated with a heightened inflammation response.

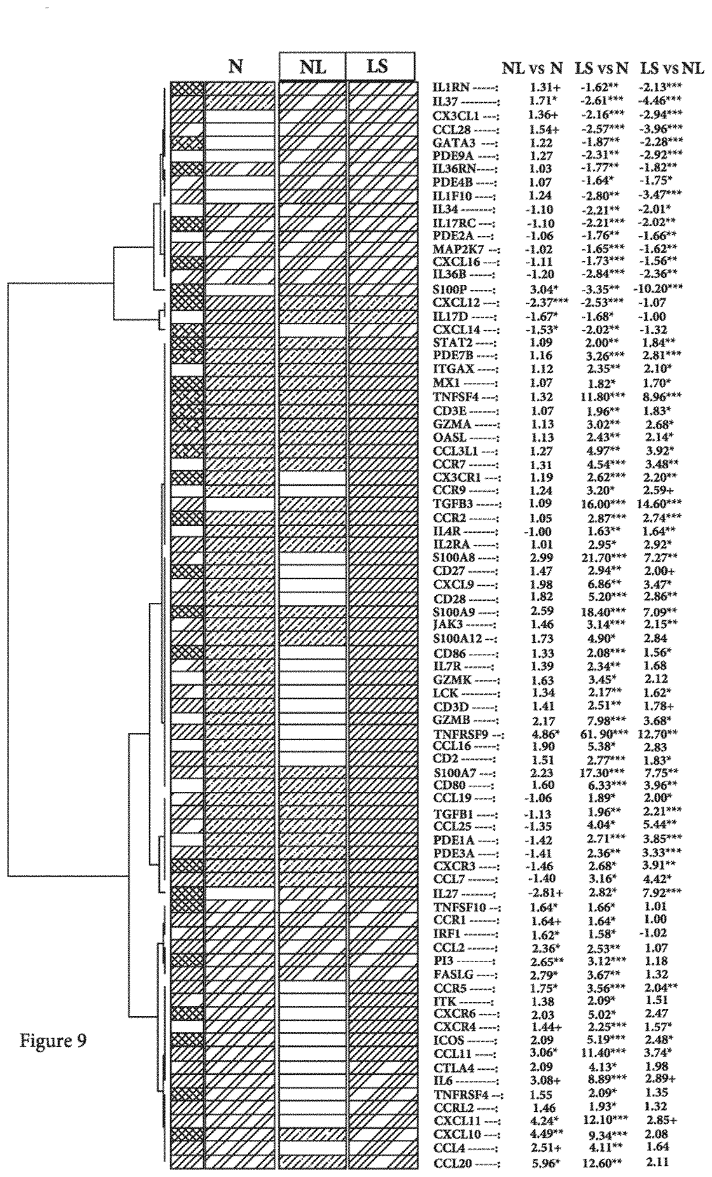

To better understand the specific inflammatory markers involved in keloids, the inventors performed RNA sequencing of lesional and non-lesional tissue in keloid patients to evaluate expression levels of certain markers correlated with Th2 activation. The data revealed activation of Th2 as demonstrated by IL4R and CCL11, TNFRSF4/OX40, TNFSF4/OX40L, CCL4, IL7R/TSLP levels. It also showed upregulation of innate immunity (IL6), Th17 (PI3, S100A8, S100A9, S100A12, CCL20) and JAK signaling (JAK3), as well as downregulation of IL34 and IL37 negative regulators (+p<0.1, *p<0.05, **p<0.01) (FIG. 9). These results further support the inventors surprising discovery that keloids arise from a heightened inflammation and particularly Th2 response rather than a mere fibrosis and wound healing response as was the prior thinking, and that they are treatable using therapies that target the inflammatory cascades involved, and particularly the Th2 inflammatory cascade.

The inventors determined that an increased dosing regimen is more effective in resolving keloids than the currently approved regimen for atopic dermatitis, where, for example, dupilumab is administered once every other week. Preferably, a regimen according to the present invention requires the active ingredient(s) be administered once per week (i.e., weekly) or more than once per week (i.e., at least weekly).

The inventors further determine that antagonists of certain inflammatory pathways will be useful for treating keloids. Drugs targeting the Janus Kinases (JAK) would be useful, including, for example: upadacitinib/JAK1 (AbbVie), abrocitinib/JAK1 (Pfizer), baricitinib JAK1/JAK2 (Eli Lilly), PF-06651600 JAK3 (Pfizer), decernotinib, filgotinib, peficitinib, PF-06700841 JAK1/TYK2 (Pfizer) and ASN002 JAK/SYK (Asana). JAK antagonists inhibit the Th2 cytokine signal transduction pathway, which includes the IL-4 and/or IL-13 signal transduction pathway, by reducing or preventing phosphorylation or dimerization of STAT transcription factors.

Also useful are IL-13 antagonists, including, for example: tralokinumab monoclonal antibody (Leo Pharma); and lebrikizumab monoclonal antibody (Dermira). Likewise, drugs directed to OX40 (a/k/a TNFRSF4) and OX40L are also useful, including, for example: KHK4083, an anti OX40 monoclonal antibody (Kyowa); GBR830, an anti OX40 monoclonal antibody (Glenmark/Ichnos Sciences); KY1005, an anti OX40L monoclonal antibody (Kymab). Drugs directed to TSLP are useful, including for example, Tezepelumab, an anti TSLP monoclonal antibody (Amgen and Astrazeneca). And drugs directed to IL-33 are useful, including, for example: REGN3500, an anti-IL-33 antagonist (Regeneron); and Etokimab, an IL-33 antagonist (Anaptysbio). And drugs targeting the IL-5 cytokine, such as mepolizumab (GSK), and Benralizumab (Astrazeneca) monoclonal antibodies.

1. A method of treating keloids comprising: administering to a subject afflicted with at least one keloid a therapeutic composition that inhibits the Th2 cytokine signaling pathway.

2. The method of claim 1, wherein the therapeutic composition comprises an antibody.

3. The method of claim 2, wherein the antibody comprises dupilumab, lebrikizumab, or tralokinumab.

4. A method of treating keloids comprising: administering to a subject afflicted with at least one keloid a therapeutic composition that inhibits IL-4, IL-13, IL-4/IL-13 cytokine, or type 2 chemokine signaling.

5. The method of claim 4, wherein the therapeutic composition comprises an antibody.

6. The method of claim 5, wherein the antibody comprises dupilumab, lebrikizumab, or tralokinumab.

7. (canceled)

8. (canceled)

9. (canceled)

10. (canceled)

11. The method of claim 4, wherein IL-4 or IL-13 signaling is inhibited.

12. The method of claim 11, wherein the therapeutic composition comprises an antibody.

13. The method of claim 12, wherein the antibody comprises dupilumab.

14. The method of claim 1, wherein the therapeutic composition comprises an antagonist of Janus Kinases (JAK).

15. The method of claim 14, wherein the composition comprises any of the following: upadacitinib, abrocitinib, baricitinib, PF-06651600, decernotinib, filgotinib, peficitinib, or ASN002.

16. A method of treating keloids comprising: administering to a subject afflicted with at least one keloid a therapeutic composition that inhibits type 2 cytokine or chemokine signaling.

17. The method of claim 16, wherein the therapeutic composition comprises an antibody.

18. The method of claim 17, wherein the antibody targets IL-13.

19. The method of claim 17, wherein the antibody targets IL-33.

20. The method of claim 17, wherein the antibody targets TSLP.

21. The method of claim 17, wherein the antibody targets IL-5.

22. The method of claim 16, wherein the therapeutic composition comprising an oral formulation that inhibits type 2 chemokine signaling.

23. The method of claim 13, wherein dupilumab is administered every week or on a weekly basis.

24. The method of claim 14, wherein the subject is not afflicted with psoriasis or atopic dermatitis.
